(19) United States
(12) Patent Application Publication
(10) Pub. No.: US 2026/0191802 A1
(43) Pub. Date: Jul. 9, 2026

(54) DRUG TO TREAT STIMULANT ADDICTION

(71) Applicant: SINGLE DOSE THERAPEUTICS, INC., Pacifica, CA (US)

(72) Inventor: Davin Rautiola, Pacifica, CA (US)

(21) Appl. No.: 19/532,903

(22) Filed: Feb. 6, 2026

Related U.S. Application Data
(63) Continuation of application No. PCT/IB2024/000660, filed on Aug. 10, 2024.
(60) Provisional application No. 63/518,550, filed on Aug. 9, 2023.

(51) Int. Cl. A61K 31/136

(52) U.S. Cl. CPC A61K 31/136

(57) ABSTRACT

Disclosed are pharmaceutical preparations comprising bromantane or an analogue of bromantane, and methods of using said preparations for pharmacotherapy to alleviate withdrawal syndrome and prevent relapse in the treatment of stimulant use disorder, to transition patients off medications that have abuse or addiction liabilities, or to facilitate extinction learning in the treatment of mental disorders.

### CROSS-REFERENCE TO RELATED APPLICATIONS

This application is a continuation application of PCT/IB2024/000660, filed Aug. 10, 2024, which claims priority to, and the benefit of U.S. Provisional Application No. 63/518,550, filed Aug. 9, 2023, the entire contents of which are incorporated herein by reference in their entirety for all purposes.

### FIELD OF INVENTION

The present invention relates to the field of medicine. Specifically, the invention involves the use of bromantane or its analogues to treat disorders of maladaptive learning.

### BACKGROUND

Central nervous system stimulants are known to be addictive. Tolerance to the effects of stimulants develops quickly and can lead to debilitating chronic dependence or overdose. Morbidity and mortality rates due to abuse of illicit stimulants (e.g., methamphetamine, cocaine) as well as misuse of licit stimulants (e.g., prescription amphetamine) have been increasing over the years, constituting a public health crisis.

Patients who are prescribed stimulant medications to treat certain neurological conditions under the supervision of a physician are also at risk of developing dependencies that can result in adverse effects which outweigh the benefits of treatment. For example, the current standard of care and first-line treatment for attention deficit disorders entails medication with either the benzylpiperazine-derived stimulant methylphenidate or phenethylamine-derived stimulant amphetamine. Patients taking these medications over a prolonged period of time tend to experience reduced efficacy, and discontinuing such prescriptions has been shown to worsen the original symptoms.

To date, no medication has been approved by the US Food and Drug Administration (FDA) for use in the treatment of Stimulant Use Disorder (StUD), commonly referred to as stimulant addiction or dependence (e.g., amphetamine dependence, cocaine dependence). In the prior art, behavioral and social interventions (e.g., cognitive behavioral therapy, contingency management) are the primary methods utilized to treat StUD; however, the success rate of those psychological approaches is moderate. Pharmacological approaches to treatment have aimed to develop drugs that target specific neurotransmitter systems such as dopaminergic, serotonergic, GABAergic, glutamatergic, adrenergic, opioidergic, or muscarinic receptors; hormonal systems such as cholecystokinin or ghrelin receptors; or metabolic pathways such as phosphodiesterases or hydroxysteroid dehydrogenase. Likewise, drugs with neuroprotective, anxiolytic, antidepressant, mood stabilizing, psychostimulant, antipsychotic, or immunotherapeutic properties have been investigated. However, a viable pharmacotherapy that significantly impacts the pathology of stimulant dependence without adverse side effects has remained elusive.

StUD and other forms of addiction are perpetuated, in part, by positive reinforcement, negative reinforcement, incentive salience, stimulus-response associations, and inhibitory control dysfunction. For example, the euphoric effects of a drug provide positive reinforcement, which increase the likelihood of repeated drug use and often drives drug-seeking behavior. Symptoms such as dysphoria or physical discomfort experienced during withdrawal from a drug are examples of negative reinforcement. In turn, psychophysiological associations are learned through these reinforcement mechanisms. Learned associations are not intrinsically adverse, but those that manifest as cravings, habits, or impulsivity can be detrimental. In the context of mental disorders such as StUD, incentive salience (‘cravings’), stimulus-response associations (‘habits’), and inhibitory control dysfunction (‘impulsivity’) are indicative of maladaptive learned associations that result in subconscious motivational states which shift attention toward counterproductive thoughts or activities, such as drug use.

Mental disorders characterized by the formation of inappropriate or dysfunctional associations between stimuli and responses can be classified as disorders of maladaptive learning. These disorders arise when normal learning processes become distorted, leading to behaviors that are harmful, excessive, or persist despite negative consequences. StUD is one such example. Maladaptive learning is challenging to rectify because learned responses tend to persist even after external reinforcement and triggers are eliminated. In other words, relapse potential remains high until the imprinted response is sufficiently unlearned. Extinction of maladaptive responses is the basis for extinction learning in the treatment of mental disorders that arise from maladaptive learning. While pharmacotherapies such as cycloserine, selective serotonin reuptake inhibitors (e.g., paroxetine, sertraline), and propranolol have shown some success in facilitating extinction learning, their efficacy is limited, and psychotherapy remains a crucial component in the treatment of disorders of maladaptive learning.

### SUMMARY OF THE INVENTION

Disclosed is the surprising discovery that the non-addictive, mild stimulant and anxiolytic drug bromantane eliminates or significantly reduces cravings and withdrawal syndrome associated with addiction to known stimulants of abuse, including but not limited to amphetamine, methamphetamine, and cocaine, without leading to secondary dependence. Furthermore, bromantane was found to simultaneously alleviate comorbidities that either arise from or exacerbate drug dependency, notably by reversing maladaptive conditioned responses. The corrective effect on the pathology of drug dependence and underlying mental disorders is rapid and remains durable after discontinuation of bromantane. Therefore, bromantane has utility as a single-agent, short-regimen, therapeutic to facilitate extinction learning in the treatment of StUD or other disorders of maladaptive learning.

Bromantane is an atypical psychostimulant that was originally investigated for its immunomodulatory, stimulant, and adaptogenic properties in the 1980's at the Research Institute of Pharmacology in Moscow, USSR. Bromantane has the capacity to boost physical endurance and was detected in Olympic athletes prior to the 1996 games, leading to its ban as a performance-enhancing drug by the World Anti-Doping Agency in 1997. Several safety and efficacy studies were conducted in Russia from 1993-2015 that demonstrated bromantane's antiasthenic properties. Oral tablets containing bromantane were approved for the treatment of neurasthenia in 2009 (Russian State Register of Medicines: LSR-010257/08). Bromantane has also been studied by various investigators around the world for use in the treatment of fibrotic diseases, cancer, liver disease, inflammation, and Parkinson's disease.

Classified as an actoprotector, bromantane is known to enhance the body's resistance to physical stress. Its pharmacology is complex, and the complete mechanism of action is still under debate in the scientific community. Its biological activity involves immunomodulation and modulation of several neurochemical processes. Bromantane is thought to produce its stimulant effects by increasing production of L-DOPA and dopamine in the hypothalamus, hippocampus, and striatum through de novo activation of tyrosine hydroxylase. Paradoxically, it also produces anxiolytic effects by inhibiting the expression of the GABA transporter gene (Gat3), enhancing GABAergic transmission by increasing GABA availability in synaptic gaps. Furthermore, it has been shown to promote neuronal survival and plasticity by causing an increase in the synthesis of effector kinases in the mitogen-activated protein kinase cascade (ERK1/ERK2) and upregulation of neurotrophic factors like brain-derived neurotrophic factor (BDNF) and nerve growth factor (NGF). The host of biological activities exhibited by bromantane is unprecedented for a single-agent pharmacotherapy in the treatment of drug dependence.

Dopaminergic activity in the brain has been implicated in the neurobiological changes that are associated with drug dependence. However, bromantane's ability to modulate dopamine cannot be considered the sole mechanism responsible for its profound and durable efficacy as an interventional therapeutic. Clinical studies of other drugs with varying degrees of specificity and selectivity to modulate dopamine in the brain have not been successful as monotherapies in the treatment of StUD. The non-selective mechanism of action of bromantane is serendipitously harmonious in terms of the broad spectrum of advantageous pharmacological effects it produces to enable extinction learning. This is an emergent property that is not predictable from what is known about the mechanism of action of bromantane or from other information available about bromantane in the prior art. Therefore, the use of bromantane to facilitate extinction learning in the treatment of drug dependence or other mental disorders is not obvious.

To investigate the utility of bromantane as a single-agent pharmacological intervention to interrupt stimulant dependence, an exploratory study was conducted with participants who had self-reported drug dependencies to a drug of concern. The intervention consisted of Test Article (bromantane) administered at a dose of 50 mg qd po for 7 days, starting on the first day of Week 1 of the study. Results from the exploratory study are summarized in [FIG. 2]. The number of days the drug of concern was used by the participant during each 1-week period during the 4-week study is shown in the Drug Dependency column. Symptom severities of comorbid conditions shown in the Comorbidity column were self-reported by each participant at the end of each week of the study using 5-point Likert rating scale where 5=symptom severity similar to severity pre-intervention, 4=slight improvement compared to pre-intervention severity, 3=significantly diminished symptoms, 2=mild symptoms, 1=no symptoms. Participants with cocaine or amphetamine dependencies achieved abstinence on the first day of intervention with the Test Article and were able to maintain abstinence for approximately three weeks. The participant with opioid dependence reported a reduction in opioid use during the period of intervention with the Test Article due to reduced ‘cravings.’ Participants also reported an unplanned reduction in caffeine use.

In addition to being an effective single-agent pharmacological intervention to interrupt stimulant dependence, it was discovered that bromantane has utility as a treatment for certain comorbidities that are specifically underpinned by maladaptive learning, such as occupational burnout exacerbated by conditioned fear responses and attention-deficit/hyperactivity disorder arising from inhibitory control dysfunction. Furthermore, participants in the exploratory study reported improved temperaments, citing a higher threshold for irritability or anger. Alleviation of insomnia caused by rumination was also noted. Analysis of the subjective effects reported by the participants revealed a pronounced reduction in counterproductive behaviors and thought patterns triggered by conditioned stimuli. This mode of action indicates that bromantane induces extinction of maladaptive stimulus-response associations, a process that can be leveraged to treat addiction as well as other mental disorders characterized by triggered responses or relapse patterns. Therefore, the present disclosure also provides novel therapeutic methods of use for bromantane to facilitate extinction learning in the treatment of mental disorders or syndromes.

Based on the observations disclosed herein for bromantane, it is anticipated that structural or functional analogues of bromantane may act similarly to induce extinction of maladaptive stimulus-response associations in humans. Therefore, the present disclosure also provides novel therapeutic methods of use for analogues of bromantane.

The objective of the present disclosure is to improve the standard of care in treating mental disorders, such as StUD, by facilitating extinction learning through the administration of a therapeutically effective amount of bromantane or an analogue thereof. While bromantane or an analogue of bromantane can be administered uncompounded, administering a pharmaceutical preparation that ensures consistent absorption and fosters patient adherence to the treatment regimen is advantageous. For example, the onset of therapeutic effect for orally administered crystalline bromantane free base is slow (2-3 hours) due to solubility-limited absorption and high first-pass extraction. Preparations that improve bromantane's solubility or bioavailability to produce a faster onset of therapeutic effect are advantageous for addressing acute incentive salience expeditiously in the treatment of StUD, thereby increasing the likelihood of patient adherence to the treatment regimen and reducing the likelihood of relapse. Accordingly, the present disclosure also provides novel, advantageous pharmaceutical preparations of bromantane and their methods of use.

### DETAILED DESCRIPTION OF THE INVENTION

The present disclosure relates to pharmaceutical preparations comprising bromantane or an analogue of bromantane, and methods of using said preparations for pharmacotherapy in the treatment of StUD or other disorders of maladaptive learning.

In one aspect of the invention, a subject who presents with symptoms of a mental disorder or syndrome is identified as having one or more features of maladaptive learning that cause, arise from, or exacerbate the subject's symptoms.

Nonlimiting examples of features of maladaptive learning include negative or positive reinforcement of harmful behaviors or thoughts, incentive salience leading to compulsive actions, maladaptive stimulus-response associations, inhibitory control dysfunction, compulsive behaviors, cognitive distortions, avoidance behaviors, emotional dysregulation, learned helplessness, conditioned fear responses, interpersonal dysfunction, response perseveration, maladaptive coping mechanisms, behavioral rigidity, or perceptual rigidity.

Maladaptive learning occurs when a subject acquires and reinforces harmful behaviors or thought patterns rather than beneficial ones, due to various biological, psychological, or environmental factors. Maladaptive learning may cause the subject to experience persistent difficulties in functioning and hinder the subject's ability to cope with life's challenges effectively. Maladaptive learning can manifest as a mental disorder that is diagnosable according to established clinical criteria (e.g., Diagnostic and Statistical Manual of Mental Disorders, International Classification of Diseases) or as a syndrome that does not meet the specific diagnostic criteria for a disorder but still involves a consistent set of maladaptive behaviors or thought patterns.

Nonlimiting examples of mental disorders in which maladaptive learning may contribute to the development, maintenance, or exacerbation of symptoms include: substance-related and addictive disorders (e.g., substance use disorders, gambling disorders), impulse control disorders (e.g., intermittent explosive disorder, kleptomania, pyromania), trauma- and stressor-related disorders (e.g., post-traumatic stress disorder, acute stress disorder, complex post-traumatic stress disorder, adjustment disorders), anxiety disorders (generalized anxiety disorder, panic disorder, social anxiety disorder, specific phobias, obsessive-compulsive disorder), mood disorders (e.g., major depressive disorder, persistent depressive disorder, bipolar disorder), neurodevelopmental disorders (e.g., attention-deficit/hyperactivity disorder), eating disorders (e.g., anorexia nervosa, bulimia nervosa, binge-eating disorder, orthorexia nervosa), personality disorders (e.g., borderline personality disorder, antisocial personality disorder, narcissistic personality disorder, obsessive-compulsive personality disorder), somatic symptom and related disorders (e.g., somatic symptom disorder, illness anxiety disorder, conversion disorder), dissociative disorders (e.g., depersonalization-derealization disorder, dissociative identity disorder), or adjustment disorder.

Nonlimiting examples of syndromes in which maladaptive learning may contribute to the development, maintenance, or exacerbation of symptoms include: drug withdrawal syndromes (e.g., serotonin syndrome, alcohol hangover, stimulant-induced anxiety, drug-induced psychosis, opioid withdrawal syndrome, rebound anxiety from withdrawal of anti-anxiety medications, post-intoxication regret, impulsive decision-making regret), stress-related syndromes (e.g., burnout, chronic fatigue), trauma-related syndromes (e.g., trauma bonding), anxiety-related syndromes (e.g., guilt complex, toxic shame, hypervigilance, hypochondriasis, separation anxiety, social anxiety, performance anxiety, fear of failure, fear of change, fear of public speaking, writers block), circadian rhythm-related syndromes (e.g., premenstrual syndrome, menopause), mood-related syndromes (e.g., affective dysregulation, dysthymia), somatization syndromes (e.g., psychogenic pain, stress-induced skin reactions), impulse control syndromes (e.g., compulsive shopping, trichotillomania, dermatillomania).

A disorder of maladaptive learning is characterized by specific deficits in learning processes, such as difficulties in extinction learning and persistent maladaptive responses to cues or triggers. Canonical mental disorders or syndromes stereotypically associated with maladaptive behaviors or thought patterns do not necessarily constitute a disorder of maladaptive learning. For example, a subject meeting diagnostic criteria for depression may exhibit symptoms due to maladaptive learning processes, such as the inability to extinguish negative thought patterns in response to certain stimuli, making their depression a disorder of maladaptive learning. However, depression can also result from other factors which do not involve maladaptive learning, such as neurobiological imbalances, genetic predispositions, or environmental stressors. The distinction between mental disorders or syndromes influenced by maladaptive learning and those same disorders or syndromes caused by other factors is important because identifying the underlying mechanisms that contribute to the development, maintenance, or exacerbation of symptoms enables the selection of correctly targeted therapeutic interventions, thereby ensuring effective treatment. The therapeutic intervention disclosed herein entails pharmacotherapy to address deficits in extinction learning that contribute to a mental disorder or syndrome, as opposed to alleviating symptomatic manifestations by other means.

In a preferred embodiment, a subject who presents with symptoms of a mental disorder or syndrome is identified as having one or more features of maladaptive learning based on clinical assessment. Clinical assessment may involve the use of scales, inventories, questionnaires, or interviews to determine the extent to which said features are responsible for the subject's symptoms, benchmark levels of distress or functional impairment, and elucidate contributing or confounding factors. Nonlimiting examples of clinical assessment tools that may be used for this purpose include Maladaptive Behavior Index (MBI), Addiction Severity Index (ASI), Yale-Brown Obsessive Compulsive Scale (Y-BOCS), Cognitive Emotion Regulation Questionnaire (CERQ), Functional Analysis Screening Tool (FAST), Beck Anxiety Inventory (BAI), Beck Depression Inventory (BDI), Minnesota Multiphasic Personality Inventory (MMPI), Behavior Assessment System for Children (BASC), Connor-Davidson Resilience Scale (CD-RISC), Structured Clinical Interview for DSM-5 (SCID-5), Obsessive-Compulsive Inventory-Revised (OCI-R), or Child Behavior Checklist (CBCL). To achieve a differential diagnosis for a disorder of maladaptive learning, criteria may be applied that distinguish a subject whose mental disorder or syndrome is primarily influenced by maladaptive learning from a subject whose mental disorder or syndrome is primarily influenced by other factors (e.g., genetic predispositions, neurobiological abnormalities, acute environmental stressors), thereby guiding targeted treatment strategies, including the use of pharmacotherapy to facilitate extinction learning.

In another preferred embodiment, a subject who presents with symptoms of a mental disorder or syndrome is identified as having one or more features of maladaptive learning based on the application of diagnostic criteria for StUD. StUD is diagnosed by clinical assessment. Nonlimiting examples of clinical assessment tools that may be used for this purpose include Structured Clinical Interview for DSM-5 (SCID-5), Addiction Severity Index (ASI), Drug Abuse Screening Test (DAST), Timeline Followback (TLFB), Substance Use Disorder Diagnostic Schedule (SUDDS), Mini International Neuropsychiatric Interview (MINI), Clinical Institute Withdrawal Assessment for Stimulants (CIWA-Stimulant), CAGE-AID Questionnaire (Adapted to Include Drugs), Composite International Diagnostic Interview (CIDI), Brief Substance Craving Scale (BSCS). A diagnosis of StUD constitutes diagnosis of a disorder of maladaptive learning. During clinical assessment, the subject's patterns of stimulant use, severity of dependence to stimulants, withdrawal symptoms, and exposure to potential relapse triggers are evaluated. Information from the evaluation is used to develop a treatment strategy involving pharmacotherapy that facilitates extinction learning to alleviate withdrawal syndrome and prevent relapse.

In some embodiments, a subject who intends to discontinue or has recently discontinued one or more medications that may have abuse or addiction liabilities is identified as having one or more features of maladaptive learning based on a risk assessment. In the case of planned discontinuation, the risk assessment considers anticipated psychological and physiological impacts of discontinuation to determine the likelihood that the subject will experience withdrawal syndrome. In the case of recent discontinuation, the risk assessment considers the severity and trajectory of acute withdrawal symptoms the subject is experiencing to determine the likelihood that the subject will develop protracted withdrawal syndrome. If the risk of the subject developing withdrawal syndrome is high, pharmacotherapy that facilitates extinction learning is administered during the transition period to mitigate withdrawal symptoms and dependence.

Prescription medications that may have abuse or addiction liabilities and are known to cause withdrawal symptoms when discontinued span a variety of drug classes, including but not limited to, antidepressants (e.g, selective serotonin reuptake inhibitors, serotonin-norepinephrine reuptake inhibitors, tricyclic antidepressants), anxiolytics (e.g., benzodiazepines, non-benzodiazepine hypnotics, beta blockers), antipsychotics, stimulants (e.g., amphetamine, methylphenidate), and pain medications (e.g. opioids, gabapentinoids).

In some embodiments, a subject who presents with symptoms of a mental disorder or syndrome is identified as having one or more features of maladaptive learning based on an empirical treatment approach. Nonlimiting examples of circumstances that may prompt this approach include those in which there is symptom alignment with a disorder of maladaptive learning, a lack of alternative treatment options, treatment resistance is observed or other treatment options have failed, greater health risk associated with other treatment options, potential benefit for adjunct therapy, or potential benefit in the treatment of one or more comorbidities. In the empirical treatment approach, pharmacotherapy that facilitates extinction learning is administered without knowing the underlying cause of the symptoms. During the first week of treatment, the subject's initial response is monitored through regular assessments of symptom changes and side effects to determine the effectiveness and safety of the pharmacotherapy. If the treatment is effective, at least some involvement of maladaptive learning processes may be inferred, and continuation of the pharmacotherapy may be indicated.

A pharmacotherapy that facilitates extinction learning enhances a subject's ability to unlearn maladaptive behaviors or thought patterns by weakening previously reinforced associations between stimuli and responses. By making it easier for the subject to disassociate learned cues from their maladaptive responses, said pharmacotherapy helps reduce the emotional and behavioral impact of triggers. This process is particularly beneficial in treating conditions in which maladaptive learning plays a critical role in symptom maintenance and relapse, such as substance use disorders, specific subsets of anxiety disorders, and specific subsets of post-traumatic stress disorder. Said pharmacotherapy comprises three key components: a pharmaceutical preparation of a therapeutic agent, a route of administration, and a dosing regimen.

In another aspect of the invention, a pharmacotherapy that facilitates extinction learning comprises a pharmaceutical preparation of a therapeutic agent, wherein the therapeutic agent is bromantane or an analogue of bromantane.

In the preferred embodiment, the therapeutic agent is bromantane [FIG. 1].

Bromantane may be synthesized using various established methods known in the art.

By way of example, one method used to synthesize bromantane is described as follows. Para-bromoformanilide (20 g, 100 mmol) was combined with bromoadamantane (10 g, 46 mmol) and stirred at 190° C. for 3 hours. The mixture was cooled to room temperature and stirring was continued as 150 mL of 13% HCl_{aq} was added. The mixture was heated again to reflux for 1 hour. Upon cooling to room temperature, 5 M NaOH was added to neutralize. Then the mixture was extracted with several portions of DCM. The extracts were pooled, washed twice with water, and dried over anhydrous Na₂SO₄. Stripping the solvent with a rotovap gave the crude product as a solid, which was then purified by recrystallization in acetonitrile. Yield of bromantane free base as a white solid was 55% (7.9 g, 26 mmol) with purity >99% by High Performance Liquid Chromatography (HPLC). Nuclear magnetic resonance (NMR): ¹H NMR (700 MHz, DMSO-d6) δ 7.15 (d, 2H), 6.58 (d, 2H), 5.73 (d, 1H), 3.41 (m, 1H), 2.02 (d, 2H), 1.89 (s, 2H), 1.81 (m, 6H), 1.70 (s, 2H), 1.48 (d, 2H), [FIG. 3]. The polymorphic form obtained was found to be thermodynamically stable and was named Form I. This form appeared as birefringent plates by polarized light microscopy (PLM) [FIG. 4], underwent melt in a hermetically sealed pan with the peak of the endothermic event occurring at 110° C. [FIG. 5], and produced a unique powder X-ray diffraction (PXRD) pattern as shown in [FIG. 6].

In some embodiments, the therapeutic agent is an analogue of bromantane. Nonlimiting examples of analogues include constitutional isomers (tautomers, regioisomers, skeletal isomers, functional group isomers, metamers, or ring-chain isomers), stereoisomers (geometric isomers, enantiomers, diastereomers, or conformational isomers), isotopes, prodrugs, metabolites, precursors, or derivatives of bromantane.

In some embodiments, an analogue of bromantane is a compound of the general structure Formula I shown in [FIG. 7]. Wherein: The molecular scaffold consists of an adamantane ring system (A) covalently linked to a benzene ring (B) by a linker (L). R₁, R₂, and R₃ are substituents on A that are independently selected from hydrogen, halogen (X=F, Cl, Br, or I), hydroxide, methoxide, methyl, or ethyl. R₄, R₅, and R₆ are substituents on B that are independently selected from hydrogen, halogen (X=F, Cl, Br, or I), hydroxide, methoxide, methyl, ethyl, or fused 5-member ring (pyrrole, furan, thiophene, imidazole, oxazole, thiazole, isoxazole, isothiazole, pyrazole, triazole, tetrazole, oxadiazole, thiadiazole, dioxole, dioxane, oxathiolane, or dithiolane). L is independently selected from amine, amide, or imine.

In some embodiments, an analogue of bromantane is a constitutional isomer (tautomer, regioisomer, skeletal isomer, functional group isomer, metamer, or ring-chain isomer), stereoisomer (geometric isomer, enantiomer, diastereomer, or conformational isomer), isotope, prodrug, metabolite, or precursor of a structure described by Formula I.

The therapeutic agent is responsible for the pharmacodynamic effect, whereas a drug substance is the physical form of the therapeutic agent used in the pharmaceutical preparation and has distinct physicochemical properties (e.g., solubility, stability) that affect pharmacokinetics (e.g., absorption rate, variability in absorption, bioavailability). The drug substance comprises the therapeutic agent and may include one or more co-formers. The distinct physicochemical properties of the drug substance arise from its morphology and the nature of the co-former. The morphology of the drug substance may be either amorphous (non-crystalline), polymorphic (thermodynamically stable or metastable crystal forms), or mesomorphic (liquid crystals).

In another aspect of the invention, the therapeutic agent is in the form of a drug substance, wherein the morphology of the drug substance is controlled and defined.

In some embodiments, the therapeutic agent is bromantane, and the form of the drug substance is a free base with Form I morphology, wherein Form I is the thermodynamically stable polymorphic crystal form.

In some embodiments, the drug substance is a salt, ionic liquid, co-crystal, hydrate, solvate, complex, conjugate, or prodrug of the therapeutic agent.

In some embodiments, the drug substance is a pharmaceutically acceptable salt or mixture of salts of bromantane. Nonlimiting examples include those wherein the pharmaceutically acceptable salt is selected from acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, tosylate, or a mixture thereof.

The formation of a salt and its physicochemical properties cannot be predicted with certainty because each potential counterion interacts uniquely with the therapeutic agent. Empirical testing is necessary. A salt screen was performed with bromantane that explored 18 counterions in 6 different solvent systems and employed a variety of crystallization methods. Results from the salt screen are shown in [FIG. 8]. Bromantane was found to form crystalline salts with the following counterions: chloride, maleate, oxalate, sulfate, methanesulfonate, phosphate, tosylate, and ethanedisulfonate.

By way of example, one method used to form a hydrochloride salt of bromantane is described as follows. The free base (1.0 g) was dissolved in ether. Dry hydrogen chloride gas was then bubbled through the solution until the precipitation of solids ceased. The solids were collected by filtration and recrystallized in ethanol. Final yield was 1.1 g of bromantane hydrochloride as a white solid, crystal Form I.

By way of example, another method used to form a hydrochloride salt of bromantane is described as follows. Ethanol (20 mL) was acidified with 150 μL of 37% HCl_{aq}. Then 0.50 g of the free base was added. The mixture was stirred until the solids were dissolved. The solution was transferred to a rotovap to strip the solvents, leaving white crystalline solids. The solids were collected, rinsed once with cold ethanol, and dried. Final yield was 0.54 g of bromantane hydrochloride, crystal Form I.

Bromantane hydrochloride had a characteristic IR band for NH₃⁺ at 3500-3400 cm⁻¹. The ionization state of the amine was also evident by NMR. ¹H NMR (400 MHz, DMSO-d6) δ 7.19 (d, 2H), 6.67 (d, 2H), 3.43 (s, 1H), 2.02 (d, 2H), 1.91 (s, 2H), 1.81 (m, 6H), 1.70 (s, 2H), 1.50 (d, 2H), [FIG. 9]. ¹H NMR (700 MHz, CDCl₃) δ 11.18 (broad s, 2H), 7.45 (d, 2H), 7.42 (d, 2H), 3.37 (s, 1H), 2.45 (d, 2H), 2.19 (s, 2H), 1.97 (s, 1H), 1.83 (m, 3H), 1.70 (s, 2H), 1.62 (d, 2H), 1.54 (d, 2H), [FIG. 10]. ¹³C NMR (176 MHz, CDCl₃) δ 143.38, 133.25, 132.63, 126.49, 123.29, 70.08, 37.22, 37.08, 30.33, 28.89, 26.97, 26.69, 18.41, [FIG. 11].

The polymorphic crystal form obtained (Form I) appeared as birefringent prisms by PLM [FIG. 4], underwent melt in a hermetically sealed pan with the peak of the endothermic event occurring at 233° C. [FIG. 12], and produced a unique PXRD pattern as shown in [FIG. 6].

By way of example, one method used to form a maleate salt of bromantane is described as follows. The free base (1.0 g) was dissolved in 5.0 mL of acetone at 50° C. Separately, 0.41 g (1.1 eq.) of maleic acid was dissolved in 5.0 mL of acetone. The acid solution was added dropwise to the solution of freebase while stirring at 250 rpm. Stirring continued while the temperature was held at 50° C. for 2 hr, during cooling to 20° C., and while the temperature was held again for 10 hr at 20° C. The resultant suspension was filtered, and the cake was dried to give bromantane maleate as a white solid, crystal Form I.

Bromantane maleate formed in a 1:1 stoichiometric ratio, as was evident by integration of the olefin peak of the counterion at δ 6.26 in the ¹H NMR spectrum. ¹H NMR (400 MHz, DMSO-d6) δ 7.16 (d, 2H), 6.58 (d, 2H), 6.26 (s, 2H), 3.41 (s, 1H), 2.03 (s, 2H), 1.89 (s, 2H), 1.81 (m, 6H), 1.70 (s, 2H), 1.49 (d, 2H), [FIG. 13]. The polymorphic crystal form obtained (Form I) appeared as birefringent needles by PLM [FIG. 4], underwent melt in a hermetically sealed pan with the peak of the endothermic event occurring at 152° C. [FIG. 14], and produced a unique PXRD pattern as shown in [FIG. 6].

Bromantane oxalate ¹H NMR (400 MHz, DMSO-d6) shifts were δ 7.15 (d, 2H), 6.58 (d, 2H), 3.41 (s, 1H), 2.03 (d, 2H), 1.89 (s, 2H), 1.81 (m, 6H), 1.70 (s, 2H), 1.49 (d, 2H), [FIG. 15]. The polymorphic crystal form obtained (Form I) appeared as birefringent granules by PLM [FIG. 4], underwent melt in a hermetically sealed pan with the peak of the endothermic event occurring at 180° C. [FIG. 16], and produced a unique PXRD pattern as shown in [FIG. 6].

Bromantane sulfate ¹H NMR (400 MHz, DMSO-d6) shifts were δ 7.19 (d, 2H), 6.65 (d, 2H), 3.43 (s, 1H), 2.01 (d, 2H), 1.90 (s, 2H), 1.81 (m, 6H), 1.70 (s, 2H), 1.50 (d, 2H), [FIG. 17]. The polymorphic crystal form obtained (Form I) appeared as birefringent granules by PLM [FIG. 4], underwent melt in a hermetically sealed pan with the peak of the endothermic event occurring at 214° C. [FIG. 18], and produced a unique PXRD pattern as shown in [FIG. 6].

Bromantane methanesulfonate formed in a 1:1 stoichiometric ratio, as was evident by integration of the peak from the methyl protons of the counterion at δ 2.34 in the ¹H NMR spectrum. ¹H NMR (400 MHz, DMSO-d6) δ 7.21 (d, 2H), 6.66 (d, 2H), 3.44 (s, 1H), 2.34 (s, 3H), 2.03 (d, 2H), 1.91 (s, 2H), 1.85 (m, 6H), 1.71 (s, 2H), 1.49 (d, 2H). The polymorphic crystal form obtained (Form I) appeared as birefringent granules by PLM [FIG. 4], underwent melt in a hermetically sealed pan with the peak of the endothermic event occurring at 214° C. [FIG. 19], and produced a unique PXRD pattern as shown in [FIG. 6].

Four polymorphic crystal forms were found for bromantane phosphate. The PXRD patterns for each form are shown in [FIG. 20]. Each form can be accessed by precipitating the salt from the appropriate solvent. The form associated with diffraction Pattern A can be obtained by precipitation from ethyl acetate. The form associated with diffraction Pattern B can be obtained by precipitation from acetone. The form associated with diffraction Pattern C can be obtained by precipitation from tetrahydrofuran. The form associated with diffraction Pattern D can be obtained by precipitation from acetonitrile.

Two polymorphic crystal forms were found for bromantane tosylate. The PXRD patterns for each are shown in [FIG. 21]. Each form can be accessed by precipitating the salt from the appropriate solvent. The form associated with diffraction Pattern A can be obtained by precipitation from either ethanol, acetone, tetrahydrofuran, or acetonitrile. The form associated with diffraction Pattern B can be obtained by precipitation from ethyl acetate.

Two polymorphic crystal forms were found for bromantane ethanedisulfonate. The PXRD patterns for each are shown in [FIG. 21]. Each form can be accessed by precipitating the salt from the appropriate solvent. The form associated with diffraction Pattern A can be obtained by precipitation from either ethanol, acetonitrile, or ethanol/water (95/5% w/w). The form associated with diffraction Pattern B can be obtained by precipitation from ethyl acetate.

The solubility advantage imparted by a specific salt form of bromantane compared to the free base form cannot be predicted with certainty because the interactions of the counterion with the therapeutic agent and dissolution medium are unique and complex. Empirical testing is necessary. Dynamic Vapor Sorption (DVS) testing was performed to compare the isothermal water sorption and desorption behaviors of bromantane free base Form I [FIG. 22] with that of bromantane hydrochloride Form I [FIG. 23]. The free base was found to be non-hygroscopic (x<0.2% w/w gain at 25° C. and 80% RH), whereas the hydrochloride salt was found to be moderately hygroscopic (2≤x<15% w/w gain at 25° C. and 80% RH). To compare aqueous solubilities, slurries of each drug substance were prepared in water, and HPLC was used to measure the solution state concentrations of bromantane that resulted in the supernatants after 24 hours of equilibration at room temperature. The equilibrium solubility of the free base in water was below the detection limit, whereas 0.2 μg/mL of solution state bromantane was measured from equilibration of the hydrochloride salt in water [FIG. 24]. Furthermore, the solution state concentration of bromantane resulting from equilibration of bromantane sulfate Form I in water was found to be 0.8 μg/mL, which is four times higher than the solubility advantage conferred by the hydrochloride salt.

In the preferred embodiment, the therapeutic agent is bromantane, and the form of the drug substance is an amorphous free base.

In some embodiments, the morphology of the drug substance is controlled by combining the drug substance with one or more pharmaceutically acceptable excipient that functions to inhibit crystallization of the drug substance (i.e., disrupt the crystalline lattice of the drug substance and stabilize the amorphous state). Nonlimiting examples of a composition comprising the drug substance and at least one excipient that functions to inhibit crystallization of the drug substance include Amorphous Solid Dispersions (ASD; e.g., spray dried dispersion, hot melt extrudates, lyophilizates, co-precipitates, amorphous granules, amorphous films, solid inclusion complexes), semi-solid or liquid solutions (e.g., gels, process feed solutions, solutions with organic vehicle components, solutions with complexing agents, lipid solutions, polymer solutions, supercritical fluids), or colloidal drug carriers (e.g. nano or micro drug suspensions, nano or micro drug carriers, micelles, liposomes, niosomes, lipid particles, emulsions, self-emulsifying drug delivery systems). Applicable excipients are selected based on their ability to inhibit crystallization of the drug substance, facilitate the drug product manufacturing process, and influence the in vivo drug release profile.

In the preferred embodiment, the therapeutic agent is bromantane, the form of the drug substance is an amorphous free base, and crystallization of the drug substance is inhibited by preparing an ASD. The ASD is prepared by mixing the drug substance with one or more pharmaceutically acceptable excipients using a suitable technique such that the drug substance is dispersed uniformly at a molecular level within the excipient matrix. Nonlimiting examples of suitable techniques to uniformly disperse the drug substance at a molecular level within the excipient matrix include solvent evaporation (e.g., spray drying, lyophilization), melt mixing (e.g., hot melt extrusion), or high shear mixing/milling/grinding. Nonlimiting examples of the ASD include those wherein the crystallization inhibiting excipient is independently selected from one or more of the following: pharmaceutically acceptable polymers (e.g., polyvinylpyrrolidone, polyvinylpyrrolidone-vinyl acetate copolymer, hydroxypropyl methylcellulose, hydroxypropyl methylcellulose acetate succinate, hydroxypropyl cellulose, dextrins, cyclodextrins, methacrylate copolymers, polyethylene glycol, poly(lactic-co-glycolic acid), polyvinyl alcohol, graft copolymer), pharmaceutically acceptable surfactants (e.g., poloxamer, polysorbate, sorbitan ester, polyoxyl castor oil, lecithin, sodium lauryl sulfate, docusate sodium, cetyltrimethylammonium bromide, benzalkonium chloride), pharmaceutically acceptable plasticizers (e.g., triethyl citrate, glycerin, propylene glycol), pharmaceutically acceptable sugars (e.g., sucrose, glucose, lactose, sorbitol, mannitol, xylitol, trehalose), pharmaceutically acceptable thermoplastics (e.g., ethylcellulose, polycaprolactone, polymethacrylate, polylactide, poly(ethylene-co-vinyl acetate)). The ratio of drug substance mass to total excipient mass is between 0.1 and 10. Selection of suitable excipients and optimization of ratios involves complementary rational design and empirical screening approaches, wherein permutations are systematically narrowed based on known physicochemical properties and theoretical interactions to reduce the number of permutations subjected to practical experimentation.

To prepare an ASD of bromantane by solvent evaporation, the drug substance and one or more crystallization inhibiting excipient are intimately mixed by dissolving both the drug substance and excipients in a volatile solvent or solvent system. Then the solvent is evaporated to recover the uniformly dispersed drug substance and excipient(s) as the ASD. Nonlimiting examples include those wherein the solvent is selected from one or more of the following: ethanol, methanol, isopropanol, acetone, dichloromethane, tetrahydrofuran, ethyl acetate, acetonitrile, methylethylketone, water, or a mixture thereof. The equilibrium solubility of bromantane free base Form I in various organic solvents is shown in [FIG. 25].

By way of example, an ASD of bromantane was prepared by solvent evaporation through spray drying as follows. A feed solution containing bromantane and copovidone at a ratio of 50/50% w/w was prepared by dissolving 6 g of copovidone and 6 g of bromantane free base Form I in 100 mL of acetone. The feed solution was aerosolized in a lab scale spray drier at a rate of 5 mL/min through a 0.5 mm atomizer at a pressure of 2 bar, with the chamber air inlet temperature set to 70° C. The ASD, recovered as white powder from the cyclone collector, was confirmed to be amorphous by PXRD [FIG. 26] and PLM.

To prepare an ASD of bromantane by melt mixing, the drug substance and one or more crystallization inhibiting excipient are mechanically mixed in a sealed vessel while heating to a temperature 2-10° C. above the transition temperature which results in a molten state. Mixing of the components in the molten state continues until a homogenous solution is observed. The homogeneous solution is then cooled to room temperature to solidify the uniformly dispersed drug substance and excipient as the ASD.

In another aspect of the invention, a pharmaceutical preparation of the therapeutic agent is a dosage form comprising a therapeutically effective amount of the drug substance and may include one or more pharmaceutically acceptable excipients. Nonlimiting examples of the dosage form include tablets, capsules, softgels, gums, pastilles, chewables, lozenges, solutions, syrups, suspensions, emulsions, ampules, granules, powders, cachets, sprays, aerosols, foams, gels, pastes, creams, suppositories, films, and patches. The excipients may include, but are not limited to, binders, fillers, diluents, disintegrants, lubricants, glidants, preservatives, stabilizers, solubilizing agents, wetting agents, emulsifiers, buffering agents, coloring agents, flavoring agents, or coating materials. Specific excipients that are suitable for each type of dosage form and route of administration would be known to a person skilled in the art of pharmaceutics.

In another aspect of the invention, a pharmaceutical preparation of the therapeutic agent is administered by one or more enteral or parenteral delivery route. The enteral delivery route involves absorption of the therapeutic agent in the gastrointestinal tract (oral—through the mouth or rectal—into the rectum). Nonlimiting examples of parenteral delivery routes include oral mucosal (e.g., sublingual, sublabial, and buccal), intranasal, inhalation (e.g., intratracheal, pulmonary), transdermal, intradermal, subcutaneous, intramuscular, intravenous, intrathecal, intraperitoneal, epidural, intravaginal, intracavernous, and intravitreal.

In the preferred embodiment, the route of administration for bromantane is oral mucosal, wherein the pharmaceutical preparation releases the therapeutic agent in the oral cavity to enable transmucosal absorption, thereby bypassing first-pass extraction and shortening the onset of therapeutic effect.

By way of example, a tablet dosage form containing 20 mg of stabilized amorphous bromantane free base was prepared. First, a 50/50% w/w ASD of bromantane in copovidone was prepared from crystalline bromantane free base Form I by spray drying as described above. The ASD was then blended at 40% w/w with 39/20/1% w/w starch, microcrystalline cellulose, and magnesium stearate. Tablets with core weights of 100 mg were formed from the blend by direct compression at 15 kN. An image of the tablet dosage form labeled as TA2 is shown in [FIG. 27].

By way of example, a tablet dosage form containing 22.4 mg of crystalline bromantane hydrochloride salt was prepared. First, the drug substance (bromantane hydrochloride salt Form I) was prepared from bromantane free base using acidified ethanol as described above. The drug substance was blended at 22.4% w/w with 39/37.6/1% w/w starch, microcrystalline cellulose, and magnesium stearate. Tablets with core weights of 100 mg were formed from the blend by direct compression at 15 kN. An image of the tablet dosage form labeled TA3 is shown in [FIG. 27].

By way of example, an oral thin film (OTF) dosage form containing 20 mg of amorphous bromantane free base was prepared. A mixture of 20/62/16/2% w/w crystalline bromantane free base Form I, pullulan, glycerin, and polysorbate was stirred in a sealed vessel while heating until a homogenous melt was observed. The warm melt was spin coated to a uniform thickness of 0.2 mm and trimmed to obtain a film with a mass of 100 mg. The film was confirmed to be amorphous, appeared isotropic (nonbirefringent) by PLM and exhibited a characteristic amorphous halo by PXRD [FIG. 26]. An image of the OTF dosage form labeled TA1 is shown in [FIG. 27].

A dissolution experiment was conducted to measure the in vitro kinetic solubility profiles of the OTF dosage form containing amorphous bromantane free base (TA1), the tablet dosage form containing an ASD of bromantane free base (TA2), and the tablet dosage form containing bromantane hydrochloride salt (TA3) that were prepared as described above. The kinetic solubility profiles were compared against that of a standard tablet dosage form containing 20 mg of crystalline bromantane free base (TA4). The standard tablet was prepared by direct compression of a 20/39/40% w/w blend of bromantane free base Form I, starch, microcrystalline cellulose, and magnesium stearate at 15 kN. The dissolution medium was simulated saliva, pH 6.8 and 37° C. The order of performance for the pharmaceutical preparations tested was TA1>TA2>TA3>TA4, indicating that the standard tablet (TA4) would produce the slowest onset of therapeutic effect and lowest bioavailability in vivo. Results from the dissolution experiment are shown in [FIG. 27].

A therapeutically effective amount (dose) of bromantane administered by oral mucosal, gastrointestinal, or a combination of oral mucosal and gastrointestinal routes ranges from 10 mg to 200 mg per day. The term therapeutically effective amount as used herein refers to the amount of an active ingredient sufficient to confer a desired prophylactic or therapeutic effect in a subject. In some embodiments, the effective amount is determined, for example, based on the administration route and frequency, body weight, and sex of the subject receiving the pharmacotherapy.

In the preferred embodiment, a method for treating a subject suffering from StUD is provided, wherein a pharmaceutical preparation of bromantane is administered as interventional pharmacotherapy. The method comprises administering bromantane to the subject at a dose ranging from 50 mg to 100 mg per day. The administration of bromantane is carried out over a treatment period of 1 to 6 weeks, depending on the severity of the disorder, the subject's response to the treatment, and the clinical judgment of the healthcare provider. During the course of treatment, the subject's progress is monitored regularly to assess the effectiveness of the intervention and to make any necessary adjustments to the dosage or duration of therapy.

### Definitions/Terms

A, an, and the: Singular articles a, an, and the are used in the text for readability, but should be understood to include plural referents unless the context clearly dictates otherwise.

Administer: As used herein, the term administer refers to the act of delivering a drug into the body of a subject by one or more delivery routes. For example, when the subject swallows a dose of the drug in the form of a tablet, the dose can be said to have been administered orally. Nonlimiting examples of delivery routes include oral, sublingual, buccal, intranasal, transdermal, or subcutaneous. Nonlimiting examples of drugs include therapeutic agents, substances of abuse (licit or illicit drug), dietary supplements, herbal remedies, or any other substances intended to affect the structure or function of the body. It is to be understood that administration of the drug can be carried out by either by the subject themselves (self-administration) or by another person, such as a healthcare professional or caregiver.

Avoidance behaviors: As used herein, the term avoidance behavior refers to an action taken to evade, escape, or reduce exposure to a perceived threat, discomfort, or distressing situation. Avoidance behaviors are learned and reinforced over time, often leading to long-term maladaptive patterns that can compound distress. A subject exhibiting avoidance behavior may go to great lengths to avoid triggers, such as skipping work to avoid stress, which can result in worsening anxiety and decreased overall functioning. Avoidance behaviors are typically negatively reinforced, meaning that the removal of a negative or distressing stimulus (e.g., anxiety, fear) strengthens the behavior. In one example, the subject exhibiting avoidance behavior may avoid a social event to reduce immediate anxiety, thereby alleviating the anxiety but reinforcing the avoidance behavior. In another example, the subject exhibiting avoidance behavior may consistently avoid public speaking, thereby negatively reinforcing the association between public speaking and anxiety, counterproductively causing the association with anxiety to become more intense and persistent over time. This negative reinforcement perpetuates maladaptive learning by making avoidance a default coping strategy.

Behavioral rigidity: As used herein, the term behavioral rigidity refers to the inflexible adherence to specific behaviors or routines, despite changing circumstances or the absence of a logical basis for such persistence. A subject exhibiting behavioral rigidity may struggle to alter their responses based on feedback or new information and may become distressed when deviations from routines or habits occur.

Cognitive distortions: As used herein, the term cognitive distortion refers to an irrational, biased, or exaggerated thought pattern that can reinforce negative emotions or behaviors. A subject experiencing cognitive distortion has a skewed perception of reality that can lead to maladaptive responses to everyday situations. Common examples include catastrophizing, overgeneralization, and personalization. In one example, the subject experiencing cognitive distortion may catastrophize (expect the worst possible outcome) social interactions, leading to avoidance of social situations and thereby reinforce social anxiety and maladaptive avoidance behaviors. In another example, the subject experiencing cognitive distortion may overgeneralize (believe that if something happens once, it will always happen) a positive experience, such as euphoria under the influence of a drug, and come to believe that using the drug will always lead to pleasurable experiences. This belief thereby reinforces drug-seeking behavior despite diminishing positive outcomes and the potential for harmful consequences. In another example, the subject experiencing cognitive distortion may personalize (misattribute causation to oneself) negative events, such as the death of a loved one, believing their actions directly caused the loss. This guilt may lead the subject to adopt maladaptive behaviors, such as withdrawing from social support or engaging in self-punishing activities, thereby reinforcing a pattern of self-blame. Cognitive distortions can also interfere with adaptive learning by preventing subjects from accurately processing information from their experiences, thereby hindering the development of effective coping strategies.

Compulsive behaviors: As used herein, the term compulsive behavior refers to a repetitive action performed to reduce or prevent distress (e.g., anxiety, sorrow, discomfort), even when the action is excessive or not logically connected to the distress. Compulsive behavior is negatively reinforced because the urge to perform the action is driven by short-term relief from distress. This negative reinforcement strengthens the behavior, making it more likely to be repeated in similar situations, thus contributing to maladaptive learning. Compulsive behaviors can generalize to other areas of life. For example, a subject who develops a compulsion related to cleanliness might start applying similar compulsive behaviors to other areas, such as checking locks or counting objects, leading to a more pervasive pattern of maladaptive behavior. Compulsive behaviors often result in long-term negative consequences, such as increased anxiety, social isolation, or impaired functioning. Recognizing compulsive behaviors involves noting actions that the subject performs with a sense of urgency or to relieve distress, such as repeated handwashing to reduce contamination fears, even when it interferes with normal life activities.

Conditioned fear responses: As used herein, the term conditioned fear response refers to an automatic response of fear or anxiety that occurs when a subject learns to associate a neutral stimulus with a fearful or traumatic event. In maladaptive learning, conditioned fear responses become ingrained and can be triggered by stimuli that pose no actual threat. The subject exhibiting conditioned fear responses may have phobic reactions or panic attacks in response to certain stimuli. For example, a subject with posttraumatic stress disorder who experienced a traumatic event in a war zone may develop a conditioned fear response to loud noises, such as fireworks, which trigger intense fear and panic attacks despite being in a safe environment.

Coping mechanisms: The terms coping mechanism, coping strategy, and coping technique are used interchangeably herein. As used herein, the term coping mechanism refers to a cognitive approach or behavioral response, whether conscious or subconscious, that a subject employs to manage stress, emotions, or difficult situations with the aim of maintaining psychological well-being. Adaptive coping mechanisms can become engrained through repeated use and reflection, thereby contributing to adaptive learning—a process by which the subject acquires or modifies behaviors, skills, or thought patterns that result in flexible, beneficial, and appropriate responses to various situations. Maladaptive coping mechanisms can become similarly engrained by providing short-term relief from stress, emotions, or difficult situations. However, maladaptive coping mechanisms do not result in flexible, beneficial, and appropriate responses in certain situations and ultimately lead to negative outcomes, exacerbating long-term distress. A subject with maladaptive coping mechanisms may resort to self-destructive behaviors to manage stress, emotions, or difficult situations, resulting in a cycle of increased distress and dysfunction. Nonlimiting examples of maladaptive coping mechanisms include substance abuse, excessive avoidance behaviors, and self-harm.

Disorder of maladaptive learning: As used herein, the term disorder of maladaptive learning refers to a condition in which a subject exhibits specific deficits in learning processes, such as difficulties in extinction learning and persistent maladaptive responses to certain stimuli. Unlike the general concept of maladaptive learning, which encompasses the acquisition or reinforcement of harmful behaviors, thoughts, or emotional responses, a disorder of maladaptive learning is characterized by a subject's chronic inability to unlearn these harmful patterns and adapt to new, healthier ones. Canonical mental disorders or syndromes that are stereotypically associated with maladaptive behaviors or thought patterns do not necessarily constitute a disorder of maladaptive learning, and a disorder of maladaptive learning does not necessarily align with these canonical mental disorders or syndromes. Treatment of a disorder of maladaptive learning requires targeted interventions aimed at extinguishing the subject's maladaptive responses. If necessary, beneficial responses can be introduced and reinforced through targeted re-learning processes, effectively replacing the maladaptive responses with healthier, adaptive ones.

Emotional dysregulation: As used herein, the term emotional dysregulation refers to the inability to manage emotional experiences in a healthy, adaptive way. Emotional dysregulation encompasses overly intense emotional responses, inappropriate emotional responses, rapidly shifting emotions, and difficulty calming down. A subject with emotional dysregulation may experience frequent mood swings, outbursts of anger, or prolonged periods of sadness or anxiety, often reacting disproportionately to events that others would consider minor. A subject with emotional dysregulation may form maladaptive associations between certain stimuli and emotional responses. Emotional dysregulation often exacerbates cognitive distortions, such as catastrophizing or dichotomous thinking, which in turn reinforces maladaptive learning. For example, intense emotions can make it more difficult to challenge irrational thoughts, leading to a cycle in which distorted thinking and emotional dysregulation reinforce each other.

Excipient: As used herein, the term excipient refers to substances, other than the therapeutic agent, that are used in pharmaceutical dosage forms. Pharmaceutically acceptable excipients are well known to those skilled in the art of pharmaceutics; and therefore, have not been listed here. A nonlimiting list of common pharmaceutically acceptable excipients can be found in the US Food and Drug Administration's Inactive Ingredient Database.

Extinction learning: As used herein, the term extinction learning refers to the process by which a subject reduces or eliminates a learned response. When the learned response is due to reinforced associations between a stimulus and a response, extinction learning involves the reduction or elimination of the association between the stimulus and the response, thereby leading to the reduction or elimination of the learned response. When the learned response occurs in the absence of the stimulus, extinction learning involves the alteration or disruption of the conscious or unconscious cognitive patterns maintaining the response, thereby leading to the reduction or elimination of the learned response. In a disorder of maladaptive learning, the learned response is maladaptive and may be a counterproductive behavior, thought pattern, or emotional reaction. Treatment of the disorder of maladaptive learning requires extinction of the maladaptive response through the process of extinction learning. Extinction learning may be facilitated by pharmacotherapy, psychotherapy, or a combination of pharmacotherapy and psychotherapy. The present disclosure provides novel pharmacotherapeutic methods to facilitate extinction learning.

Feature of maladaptive learning: As used herein, the term feature of maladaptive learning refers to an identifiable characteristic, behavior, or cognitive pattern exhibited by a subject that indicates the presence of maladaptive learning processes. Features of maladaptive learning in the subject may involve maladaptive responses to stimuli, reinforcement of harmful behaviors or thoughts, dysfunctional associations between stimuli and outcomes, or impairments in cognitive or emotional regulation that cause or result from maladaptive coping mechanisms.

Incentive salience: As used herein, the term incentive salience refers to the desirability that a subject attributes to a stimulus based on its association with a rewarding outcome. The subject experiences incentive salience as craving or wanting the stimulus. Unlike the sensation of liking, which is the immediate pleasure gained from a rewarding stimulus, incentive salience refers to a motivational state in which the sensation of craving or wanting drives the subject to seek out the stimulus. Attribution of unfounded or exaggerated desirability to the stimulus can adversely affect motivational prioritization and distort cognitive processes, causing the subject to irrationally believe that obtaining the desired stimulus is essential for their well-being or survival, thereby overriding logical decision-making. Incentive salience can also reinforce and perpetuate harmful behavior or thought patterns, making them more automatic and difficult to change, leading to compulsions such as addiction or obsessive routines.

Inhibitory control dysfunction: As used herein, the term inhibitory control dysfunction refers to the impaired ability to suppress inappropriate or unwanted behaviors, thoughts, or emotions. A subject with inhibitory control dysfunction struggles to regulate their impulses or refrain from actions that are detrimental to themselves or others. The subject may act on harmful impulses without considering the consequences, such as reacting with aggression during minor conflicts or failing to resist the urge to engage in risky behaviors. The subject may struggle to suppress maladaptive behaviors once they have been learned. For example, the subject might repeatedly engage in substance use or other impulsive actions because they cannot effectively inhibit these behaviors despite experiencing negative outcomes. When faced with stressful situations or triggers, the subject may default to maladaptive behaviors due to a lack of self-regulation needed to implement healthier coping strategies. Inhibitory control dysfunction often leads to a preference for immediate gratification over long-term benefits. This preference reinforces maladaptive learning because the subject is more likely to engage in behaviors that provide short-term relief or pleasure, such as overeating, substance use, or compulsive shopping, rather than behaviors that require delayed gratification.

Interpersonal dysfunction: As used herein, the term interpersonal dysfunction refers to difficulties in forming and maintaining healthy relationships. Interpersonal dysfunction is often rooted in learned negative interaction patterns and reinforced by maladaptive responses to interpersonal conflict. A subject with interpersonal dysfunction may struggle with trust, communication, or empathy, leading to arguments, social isolation, and challenges in building supportive relationships.

Learned helplessness: As used herein, the term learned helplessness refers to a psychological state in which a subject has learned to believe they are powerless to change their situation due to repeated exposure to uncontrollable events. Learned helplessness affects the subject's motivation, cognition, and emotional responses, leading the subject to believe they have no control over the outcomes of their actions, even when control is possible. In disorders of maladaptive learning, learned helplessness can become entrenched, leading to passivity and a lack of initiative. The subject with learned helplessness may exhibit signs of resignation and avoidance of challenges because they believe their actions will not make a difference, which can perpetuate a cycle of inactivity and depression. The subject with learned helplessness may express the belief that their actions are futile, complain of low motivation, or exhibit symptoms of depression or anxiety when faced with challenges.

Maladaptive learning: As used herein, the term maladaptive learning refers to the process by which a subject acquires or reinforces behaviors, thoughts, or emotional responses that are detrimental to their well-being or functioning. Maladaptive learning may occur through the reinforcement of harmful behaviors or thoughts, dysfunctional associations between stimuli and outcomes, or impairments in cognitive and emotional regulation that result in maladaptive coping mechanisms. Maladaptive learning leads to patterns that are inflexible or counterproductive, often exacerbating stress, anxiety, and other psychological issues.

Negative reinforcement: As used herein, the term negative reinforcement refers to the strengthening of a behavior or thought pattern through the removal of an unpleasant stimulus. Often this type of reinforcement affects behaviors such as engaging in a particular action to avoid or stop an adverse condition. For example, a behavior of self-harm is reinforced in a subject who is engaging in self-harm to relieve emotional pain or taking painkillers to relieve a headache. As used herein, negative reinforcement also encompasses the strengthening of avoidance behaviors through association with unpleasant stimuli. This means that the presence of an unpleasant stimulus can increase the likelihood of certain associated behaviors or situations being avoided in the future to prevent experiencing the unpleasant stimulus again.

Perceptual rigidity: As used herein, the term perceptual rigidity refers to the inflexible adherence to specific interpretations or thought patterns, despite changing circumstances or the absence of a logical basis for such persistence. A subject exhibiting perceptual rigidity may struggle to alter their interpretations based on new information or feedback and may become distressed when confronted with perspectives or evidence that challenge their fixed viewpoints. In maladaptive learning, perceptual rigidity results in a fixed, often negative view of oneself, others, or the world. The subject with perceptual rigidity may persistently view themselves as incapable or unworthy despite evidence to the contrary, or maintain a distorted view of social interactions, leading to persistent interpersonal conflicts and self-esteem issues.

Pharmaceutically acceptable: As used herein, the term pharmaceutically acceptable refers to substances used in a drug product that are considered generally safe (i.e., non-toxic and otherwise biocompatible in the manner and amounts employed, commensurate with a reasonable benefit/risk assessment) for human pharmaceutical use.

Positive reinforcement: As used herein, the term positive reinforcement refers to the strengthening of a behavior or thought pattern through the presentation of a pleasant or rewarding stimulus. Often this type of reinforcement affects behaviors. Subjects may learn and perpetuate harmful or non-beneficial behaviors when those behaviors are inadvertently reinforced by positive outcomes. In substance use disorder, positive reinforcement occurs when the pleasurable effects of a drug encourage continued use, thereby reinforcing the behavior of drug consumption. Positive reinforcement encompasses the introduction of any desirable outcome that increases the likelihood of a behavior being repeated, whether it be tangible rewards, praise, or any other form of positive feedback that encourages continuation of the behavior.

Response: As used herein, response refers to any behavioral, emotional, or cognitive reaction elicited by a stimulus, encompassing both conscious and unconscious actions or changes in a subject's thought patterns that result from exposure to an external or internal event, object, or condition.

Response perseveration: As used herein, the term response perseveration refers to the repetition of a particular response despite the absence or cessation of a stimulus. A subject exhibiting response perseveration may have difficulty switching thoughts or behaviors in response to changing situations. The subject exhibiting response perseveration may ruminate (e.g., continuously dwelling on a specific worry) or continue a specific action long after the relevance of the stimulus has ended (e.g., compulsive actions such as dermatillomania), demonstrating inflexibility in thought or behavior.

Stimulus: As used herein, stimulus or its plural form, stimuli, refers to any external or internal event, object, or condition that elicits a psychological response (i.e., behavioral, emotional, or cognitive reaction) from a subject. Stimulus is used herein synonymously with trigger and cue. These terms are used interchangeably to describe factors that can elicit or modify the subject's response patterns.

Stimulus-response associations: As used herein, the term stimulus-response associations refers to learned connections in which specific environmental triggers or cues (stimuli) elicit specific behavioral, emotional, or cognitive reactions (response), often leading to automatic or habitual patterns of action and corresponding reaction. These entrenched patterns are resistant to alteration, even when harmful. Maladaptive stimulus-response associations hinder learning new, healthier responses and interfere with the extinction of dysfunctional responses. For example, overgeneralization of responses to various stimuli can lead to reinforcement of adverse thought patterns that may manifest as or exacerbate mental disorders such as anxiety or depression. Subjects suffering from anxiety may develop associations between certain situations and fear responses, leading to avoidance behaviors that perpetuate the anxiety. Recognition of maladaptive stimulus-response associations in a subject involves observing repetitive, automatic responses to specific stimuli that do not seem to change despite negative outcomes or attempts at intervention.

Subject: As used herein, the term subject refers to any human. The terms subject, patient, individual, and person may be used interchangeably herein. When the subject has taken part in or is taking part in a research study, the term participant may also be used. The terms subject, patient, individual, person, and participant include those who have symptoms of a mental health disorder, a substance use disorder, or a condition related to the same, whether or not the subject is diagnosed with such a disorder. Moreover, these terms shall likewise refer to subjects who have received treatment or therapy in the past, are currently receiving treatment or therapy, or who may receive treatment or therapy in the future for a mental health disorder or substance use disorder. The disclosed methods of treatment can be modified to treat multiple subjects at once, including couples, families, or groups. Hence, these terms will be understood to also mean two or more subjects.

Symptom: As used herein, the term symptom refers to any physical or mental abnormality experienced or exhibited by a subject. Symptoms can vary in origin, intensity, duration, and impact on the subject's daily functioning. The term symptom encompasses clinical manifestations that indicate the presence of a disease, disorder, or syndrome. The term symptom also encompasses clinical manifestations that indicate a disease, disorder, or syndrome may develop.

Syndrome: As used herein, the term syndrome refers to a set of symptoms (i.e., a consistent and recognizable pattern of clinical manifestations) experienced or exhibited by a subject that characterize a certain abnormal state of physical or mental health, such as a disease or disorder. The term syndrome encompasses symptomology that indicates the subject may have an increased chance of developing a disease or disorder. The syndrome may be associated with a specific underlying cause or may represent a group of related causes without a single identifiable origin.

Test Article: As used herein, the term Test Article is a moniker for the active agent being tested and is used to enable blinding.

While the invention is described in terms of particular embodiments and applications, it is not intended that these descriptions in any way limit its scope to any such embodiments and applications, and it will be understood that many modifications, substitutions, changes, and variations in the described embodiments, applications, and details of the invention can be made by those skilled in the art without departing from the spirit or scope of the invention. Similarly, the exemplary embodiments illustrated in referenced figures are intended to be illustrative rather than restrictive. The aim of this disclosure to provide an understanding of the invention to persons skilled in the art. Well known elements are not described in detail to avoid unnecessarily obscuring the disclosure.

### BRIEF DESCRIPTION OF THE FIGURES

FIG. 1 illustrates the chemical structure of bromantane.

FIG. 2 is a table showing the results from an exploratory study that tracked the effects of pharmacological intervention with bromantane on participants' frequency of drug use and symptoms of comorbid conditions.

FIG. 3 is a graph showing the ¹H NMR spectrum of bromantane free base in DMSO-d6.

FIG. 4 is a series of PLM images showing the birefringence of bromantane free base Form I, bromantane hydrochloride salt Form I, bromantane maleate salt Form I, bromantane oxalate salt Form I, bromantane sulfate salt Form I, and bromantane methanesulfonate salt Form I.

FIG. 5 is a graph showing a differential scanning calorimetry (DSC) thermogram of bromantane free base Form I.

FIG. 6 is a graph showing the PXRD patterns of bromantane oxalate salt Form I, bromantane maleate salt Form I, bromantane methanesulfonate salt Form I, bromantane sulfate salt Form I, bromantane hydrochloride salt Form I, and bromantane free base Form I.

FIG. 7 is a table illustrating the general chemical structure of Formula I, the substituent pattern that constitutes bromantane, and the various substituent substitution permutations that represent analogues of bromantane.

FIG. 8 is a table showing the results from a bromantane salt screening study.

FIG. 9 is a graph showing the ¹H NMR spectrum of bromantane hydrochloride salt in DMSO-d6.

FIG. 10 is a graph showing the ¹H NMR spectrum of bromantane hydrochloride salt in CDCl₃.

FIG. 11 is a graph showing the ¹³C NMR spectrum of bromantane hydrochloride salt in CDCl₃.

FIG. 12 is a graph showing the DSC thermogram of bromantane hydrochloride salt Form I.

FIG. 13 is a graph showing the ¹H NMR spectrum of bromantane maleate salt in DMSO-d6.

FIG. 14 is a graph showing the DSC and thermogravimetric analysis (TGA) thermograms of bromantane maleate salt Form I.

FIG. 15 is a graph showing the ¹H NMR spectrum of bromantane oxalate salt in DMSO-d6.

FIG. 16 is a graph showing the DSC and TGA thermograms of bromantane oxalate salt Form I.

FIG. 17 is a graph showing the ¹H NMR spectrum of bromantane sulfate salt in DMSO-d6.

FIG. 18 is a graph showing the DSC and TGA thermograms of bromantane sulfate salt Form I.

FIG. 19 is a graph showing the DSC thermogram of bromantane methanesulfonate salt Form I.

FIG. 20 is a graph showing the PXRD patterns of four distinct crystalline polymorphs of the bromantane phosphate salt.

FIG. 21 is a graph showing the PXRD patterns of two distinct crystalline polymorphs of the bromantane p-tosylate salt and two distinct crystalline polymorphs of the bromantane ethanedisulfonate salt.

FIG. 22 is a graph showing the isothermal water sorption and desorption behaviors of bromantane free base Form I.

FIG. 23 is a graph showing the isothermal water sorption and desorption behaviors of bromantane hydrochloride salt Form I.

FIG. 24 is a table showing the aqueous equilibrium solubilities measured for bromantane free base Form I, bromantane maleate salt Form I, bromantane oxalate salt Form I, and bromantane sulfate salt Form I.

FIG. 25 is a table showing the equilibrium solubility of bromantane free base Form I in organic solvents.

FIG. 26 is a graph showing the PXRD patterns of bromantane free base ASD in copovidone, bromantane free base OTF dosage form, and bromantane carboxymethyl-3-cyclodextrin inclusion complex.

FIG. 27 is a graph showing the results of a dissolution experiment comparing the kinetic solubility profiles of various pharmaceutical preparations of bromantanes.

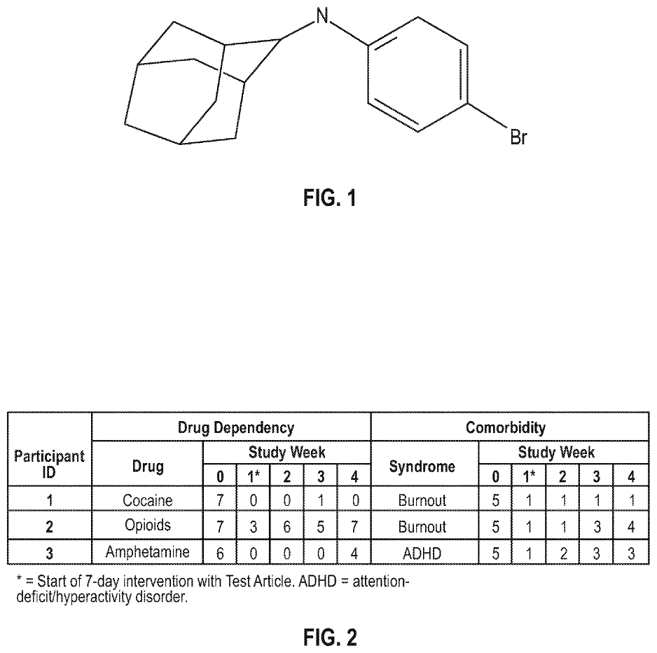

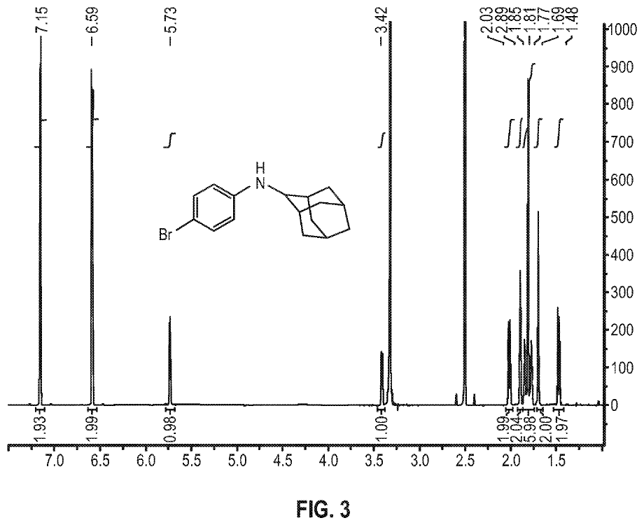

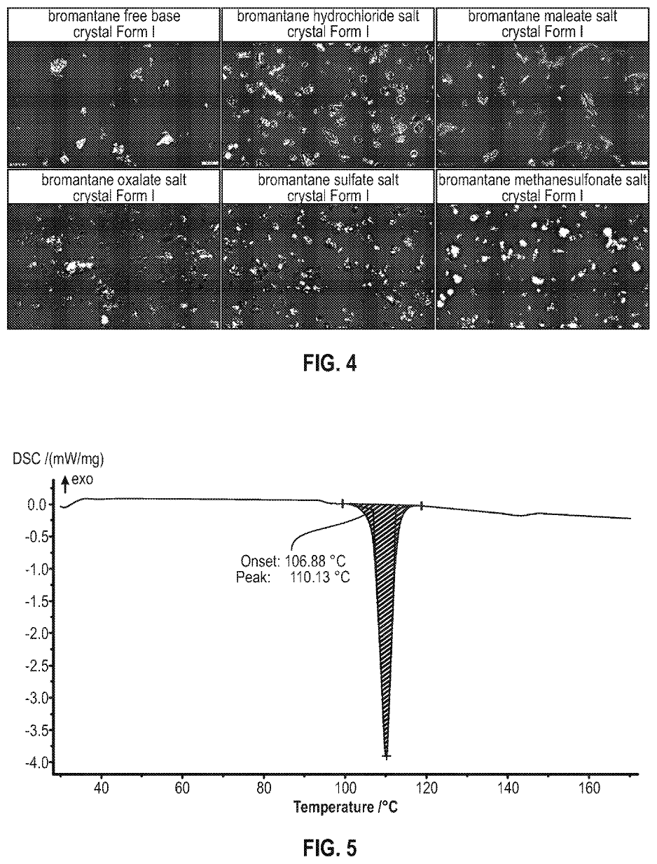

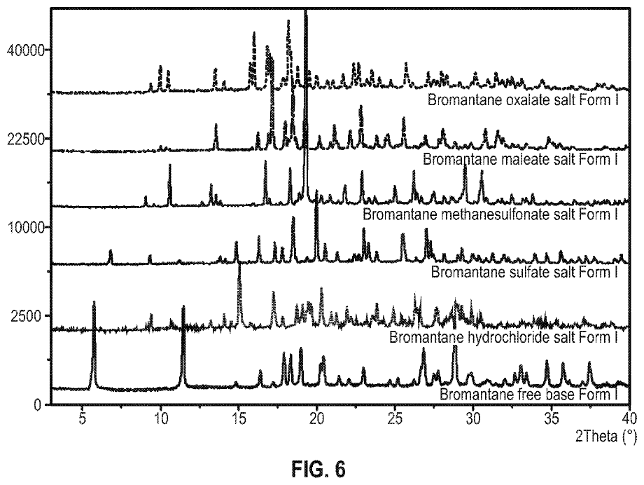

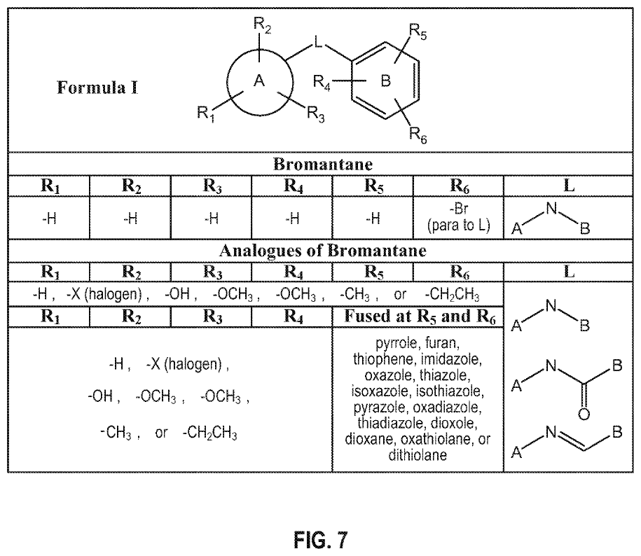

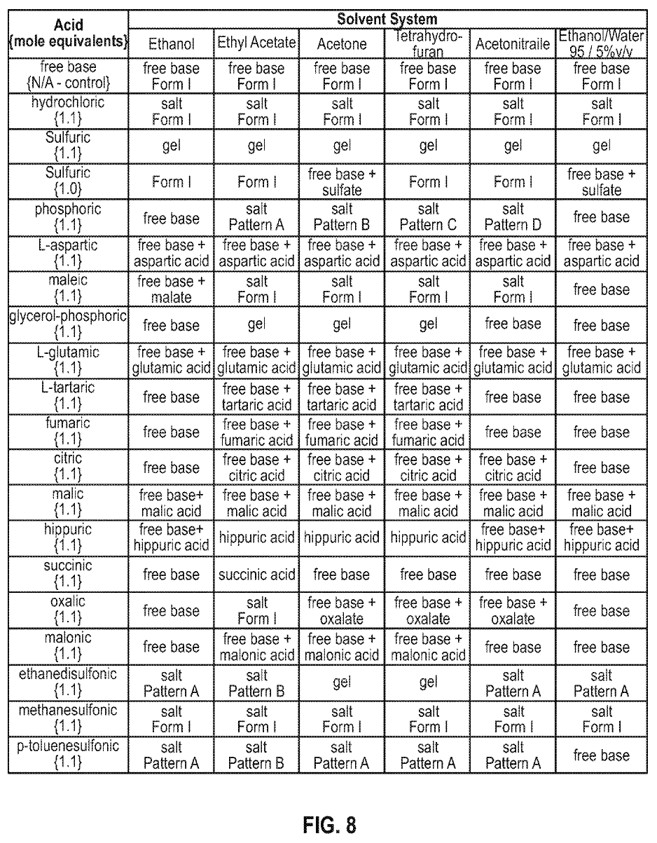

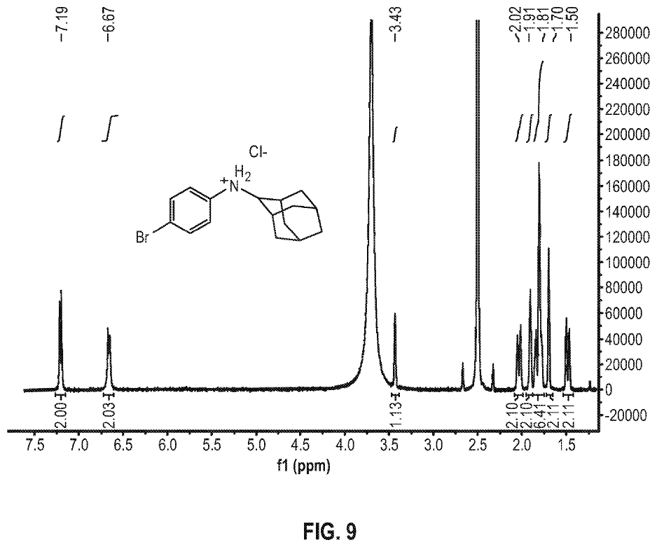

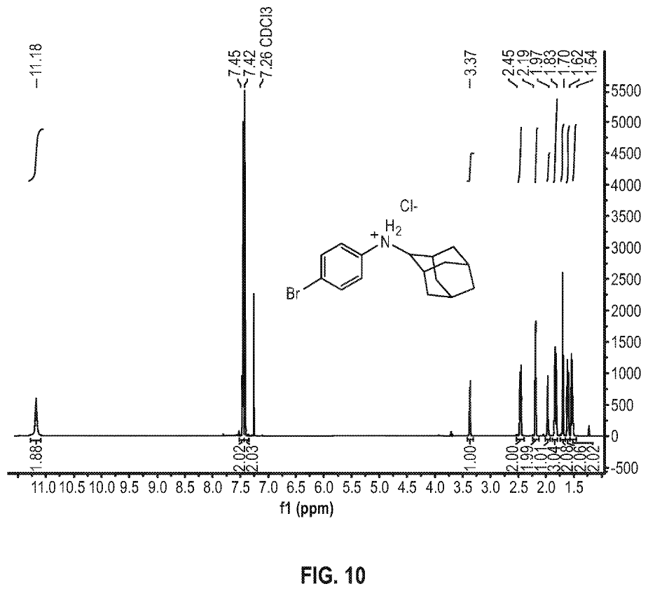

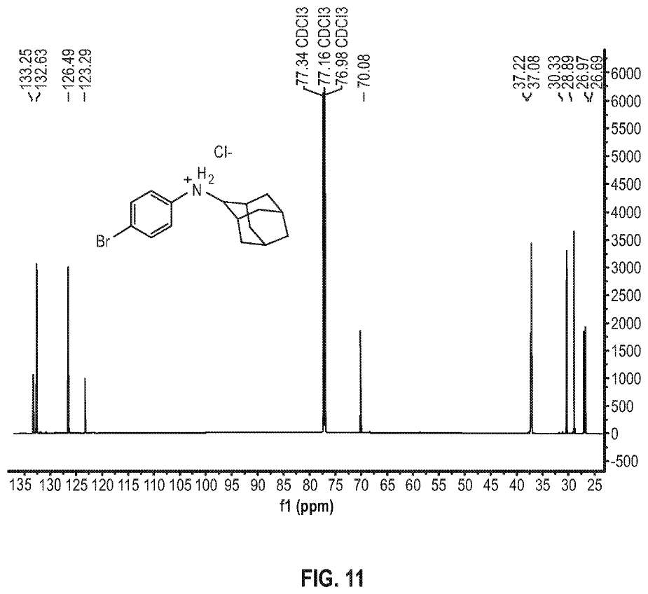

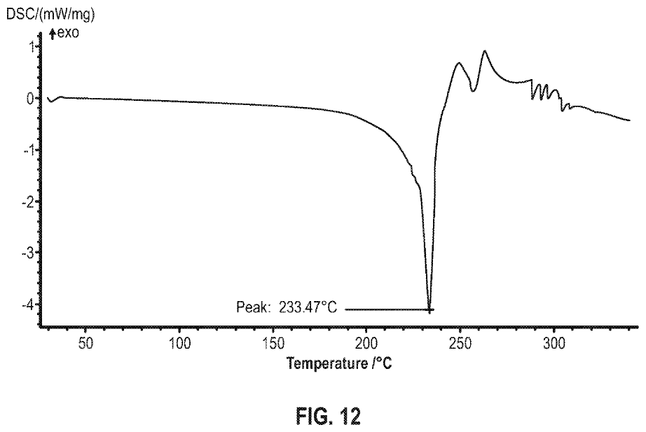

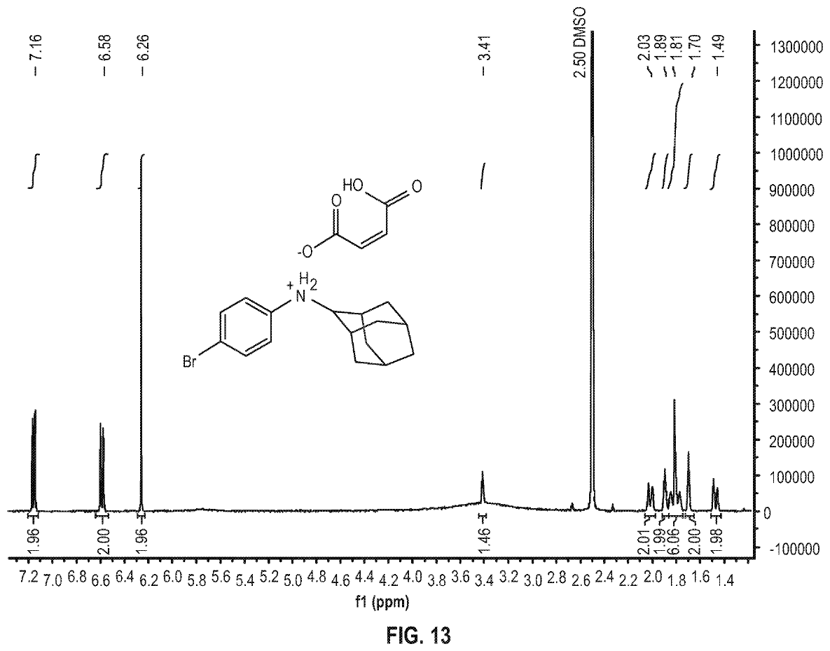

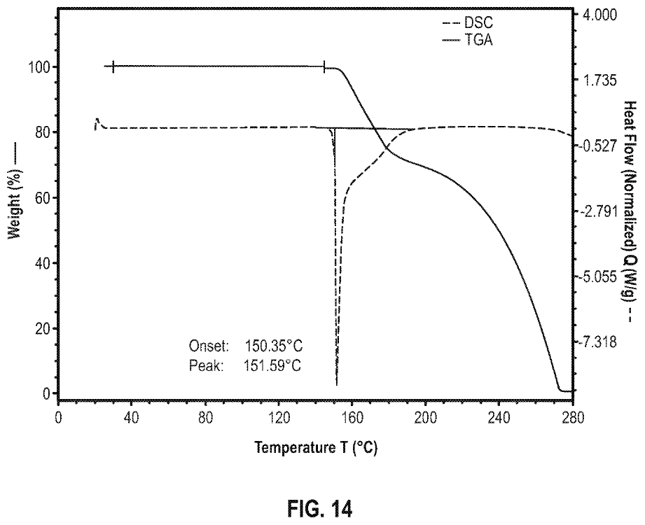

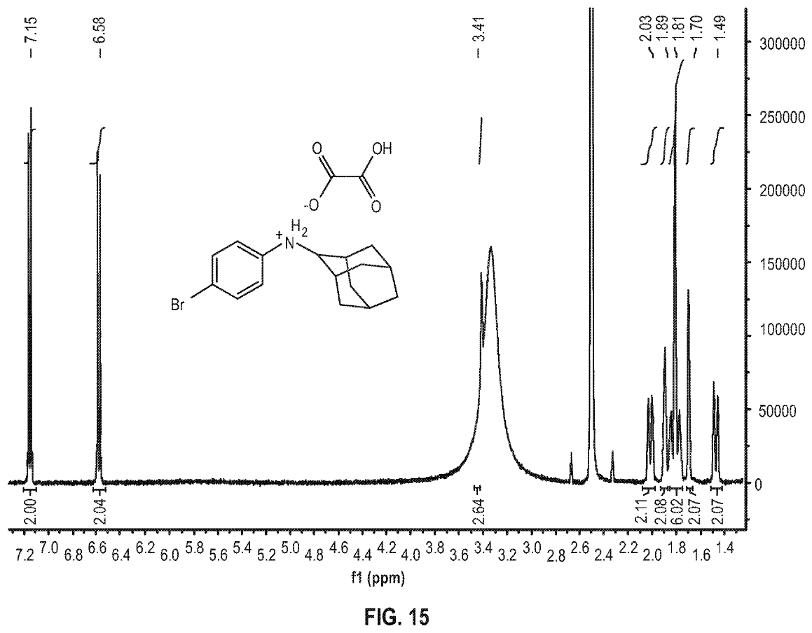

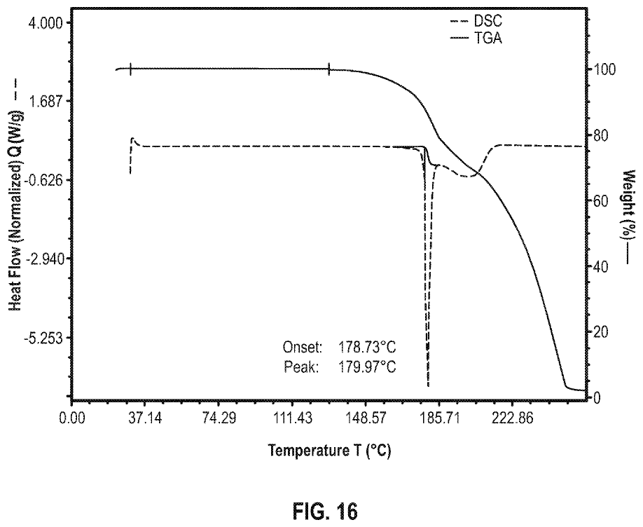

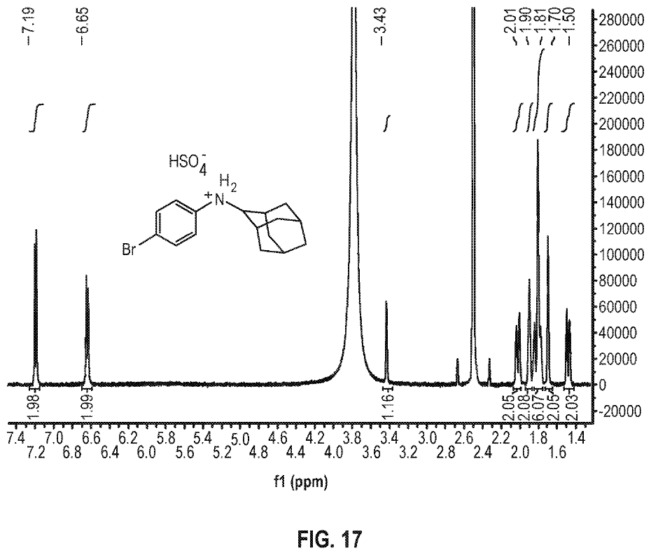

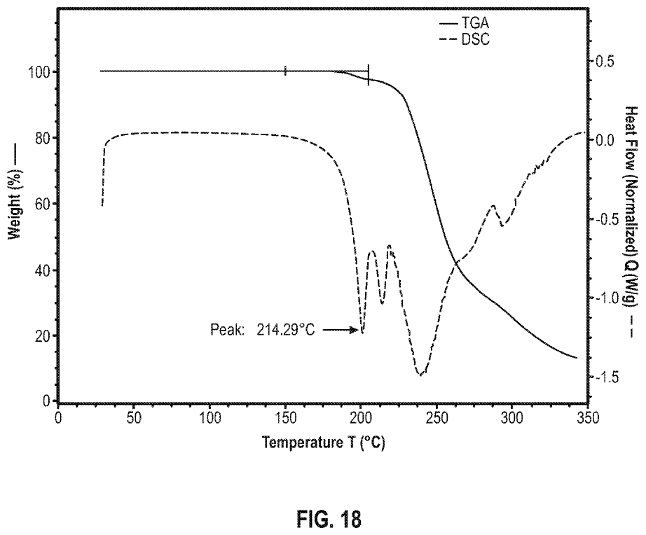

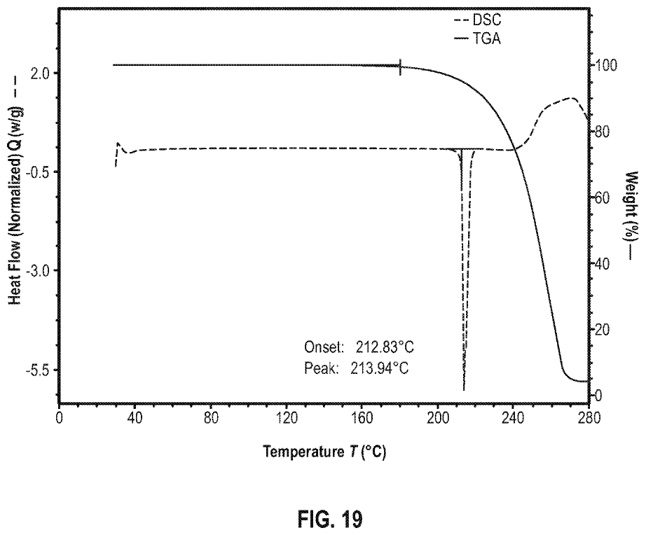

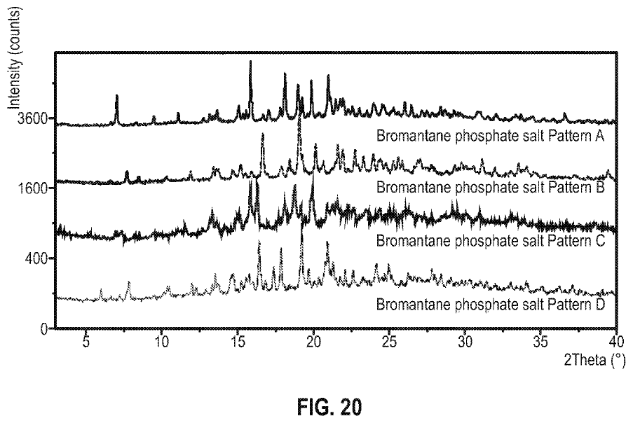

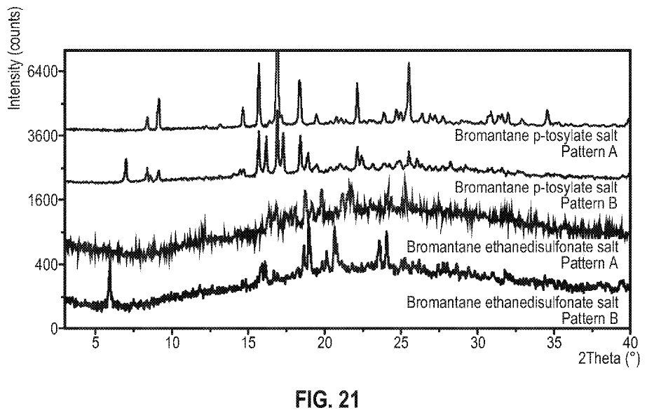

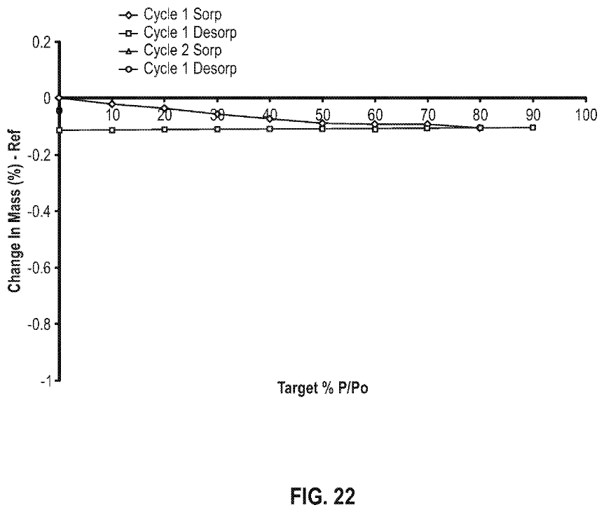

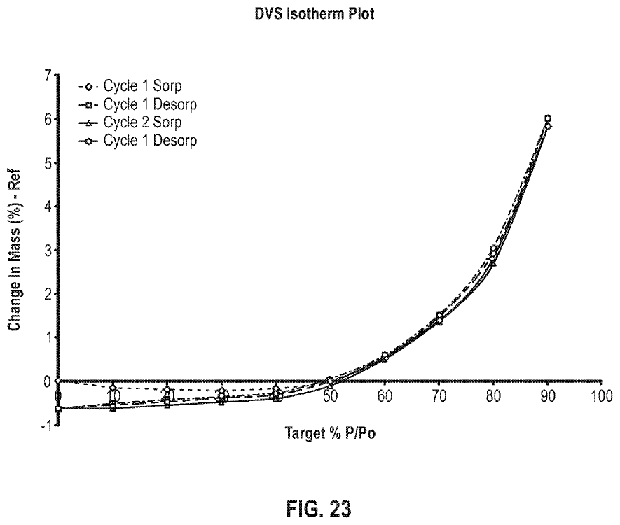

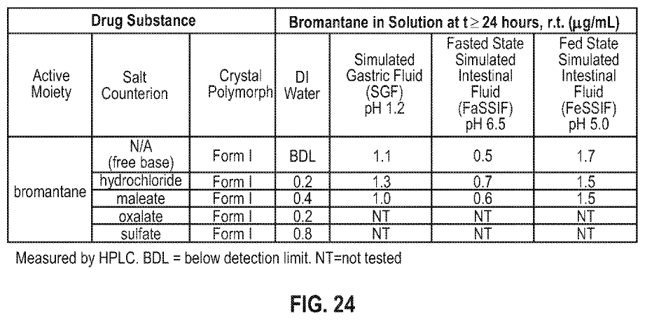

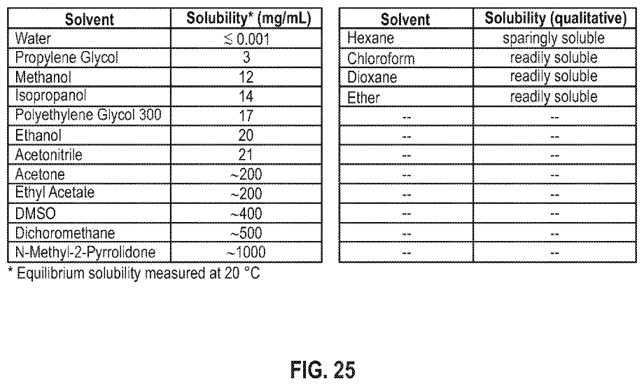

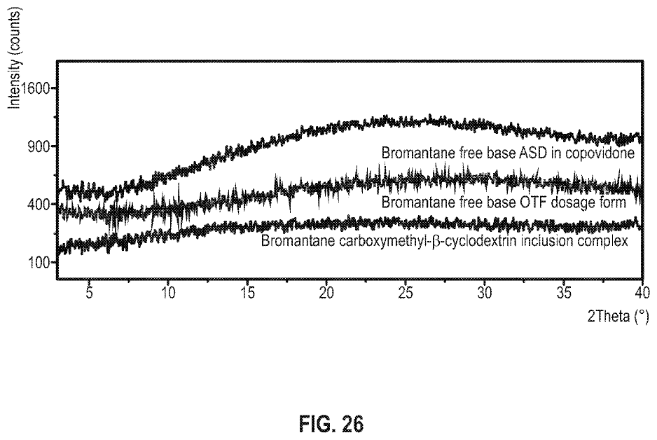

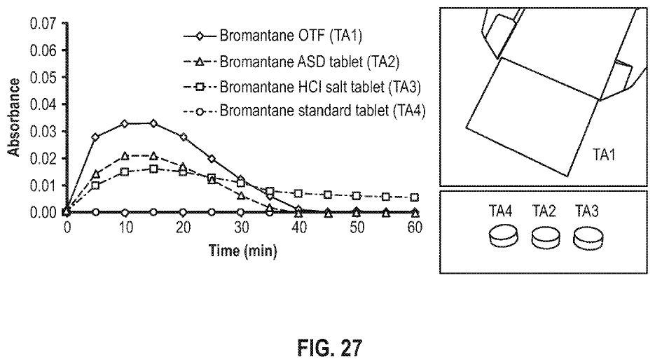

1-8. (canceled)

9. A method of treating a disorder of maladaptive learning in a subject in need thereof, the method comprising:
facilitating extinction learning in the subject in need thereof by administering a therapeutically effective amount of a pharmaceutical preparation of bromantane, or an analogue of bromantane, to the subject in need thereof.

10. The method of claim 9, wherein the therapeutically effective amount is a dose ranging from 10 mg to 200 mg per day.

11. The method of claim 9, wherein the therapeutically effective amount is administered only once per day and without any administering of a secondary therapeutic agent.

12. The method of claim 9, wherein the therapeutically effective amount is 50 mg and administered orally once a day for 7 days.

13. The method of claim 9, wherein the therapeutically effective amount is a dose ranging from 50 mg to 100 mg per day, wherein the administering is carried out over a treatment period of 1 week to 6 weeks.

14. The method of claim 9, wherein the disorder of maladaptive learning is a drug withdrawal syndrome.

15. The method of claim 14, wherein the drug withdrawal syndrome is a stimulant withdrawal syndrome.

16. The method of claim 9, wherein the disorder of maladaptive learning is a substance use disorder.

17. The method of claim 16, wherein the substance use disorder is stimulant use disorder.

18. The method of claim 9, wherein the disorder of maladaptive learning is intermittent explosive disorder, kleptomania, pyromania, post-traumatic stress disorder, acute stress disorder, complex post-traumatic stress disorder, adjustment disorders, generalized anxiety disorder, panic disorder, social anxiety disorder, phobias, obsessive-compulsive disorder, major depressive disorder, persistent depressive disorder, bipolar disorder, anorexia nervosa, bulimia nervosa, binge-eating disorder, orthorexia nervosa, borderline personality disorder, antisocial personality disorder, narcissistic personality disorder, obsessive-compulsive personality disorder, somatic symptom disorder, illness anxiety disorder, conversion disorder, depersonalization-derealization disorder, dissociative identity disorder, or combinations thereof.

19. The method of claim 9, wherein bromantane, or the analogue of bromantane, has a chemical structure according to Formula I, 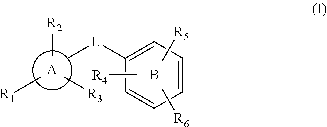
wherein an adamantane ring system (A) is covalently linked to a benzene ring (B) by a linker (L);
R₁, R₂, and R₃ are independently selected from hydrogen, fluorine, chlorine, bromine, iodine, hydroxide, methoxide, methyl, or ethyl;
R₄, R₅, and R₆ are independently selected from hydrogen, fluorine, chlorine, bromine, iodine, hydroxide, methoxide, methyl, ethyl, or fused 5-member ring, wherein the fused 5-member ring is selected from pyrrole, furan, thiophene, imidazole, oxazole, thiazole, isoxazole, isothiazole, pyrazole, triazole, tetrazole, oxadiazole, thiadiazole, dioxole, dioxane, oxathiolane, or dithiolane;
L is independently selected from amine, amide, or imine.

20. The method of claim 19, wherein L is amine.

21. The method of claim 19, wherein L is amide.

22. The method of claim 19, wherein L is imine.

23. The method of claim 19, wherein at least one of R₁, R₂, and R₃ is fluorine, chlorine, bromine, iodine, hydroxide, methoxide, methyl, or ethyl.

24. The method of claim 9, wherein the pharmaceutical preparation is in the form of a tablet.

25. The method of claim 9, wherein the pharmaceutical preparation comprises one or more pharmaceutically acceptable excipients that inhibits crystallization.

26. The method of claim 9, wherein the bromantane, or the analogue of bromantane is amorphous.

27. The method of claim 9, further comprising prior to administering the therapeutically effective amount, assessing a subject suffering from symptoms of a mental disorder or syndrome to determine the presence of one or more features of maladaptive learning that cause, arise from, or exacerbate the symptoms.

28. The method of claim 9, wherein the analogue of bromantane is selected from constitutional isomers, stereoisomers, isotopes, prodrugs, metabolites, precursors, or derivatives of bromantane.

29. The method of claim 28, wherein the analogue of bromantane is constitutional isomers selected from tautomers, regioisomers, skeletal isomers, functional group isomers, metamers, ring-chain isomers, or combinations thereof.

30. The method of claim 28, wherein the analogue of bromantane is stereoisomers selected from geometric isomers, enantiomers, diastereomers, conformational isomers, or combinations thereof.

31. The method of claim 9, wherein the bromantane, or the analogue of bromantane, is an amorphous free base.

32. The method of claim 9, wherein the pharmaceutical preparation is a tablet containing 20 mg of stabilized amorphous bromantane.

33. The method of claim 9, wherein the pharmaceutical preparation is a tablet containing 22.4 mg of crystalline bromantane hydrochloride salt.
